(19) United States
(12) Patent Application Publication
(10) Pub. No.: US 2021/0205409 A1
(43) Pub. Date: Jul. 8, 2021

(54) METHOD FOR THE TREATMENT OF HYPOXIC-ISCHEMIC ENCEPHALOPATHY IN NEWBORNS

(71) Applicants: UNIVERSITEIT MAASTRICHT, MAASTRICHT (NL); ACADEMISCH ZIEKENHUIS MAASTRICHT, MAASTRICHT (NL)

(72) Inventors: Tim Guillaume Anna Marie WOLFS, MAASTRICHT (NL); Christiaan Peter Maria REUTELINGSPERGER, MAASTRICHT (NL)

(21) Appl. No.: 17/059,877

(22) PCT Filed: Jun. 7, 2019
(86) PCT No.: PCT/EP2019/064914

§ 371 Date: Nov. 30, 2020

(30) Foreign Application Priority Data
Jun. 12, 2018 (EP) 18177287.2

(51) Int. Cl. A61K 38/17; A61P 25/00

(52) U.S. Cl. CPC A61K 38/1709; A61P 25/00

(57) ABSTRACT

The invention is in the field of medical treatments. It provides means and methods for treating acute or sub-acute brain injury due to asphyxia in newborns. It has now been found that hypoxic-ischemic encephalopathy may effectively be treated by administering a composition comprising Annexin A1 to a subject in need of such a treatment. The invention therefore relates to a treatment for hypoxic-ischemic encephalopathy in newborns by administering a composition comprising Annexin A1 to a preterm born newborn.

### FIELD OF THE INVENTION

The invention is in the field of medical treatments. It provides means and methods for treating acute or sub-acute brain injury due to asphyxia in preterm newborns. More in particular, it provides a treatment for hypoxic-ischemic encephalopathy.

### BACKGROUND OF THE INVENTION

Perinatal asphyxia, more appropriately known as hypoxic-ischemic encephalopathy (HIE), is characterized by clinical and laboratory evidence of acute or subacute brain injury due to asphyxia. The primary causes of this condition are systemic hypoxemia and/or reduced cerebral blood flow (CBF). Birth asphyxia causes 840,000 or 23% of all neonatal deaths worldwide [1, 2, 3].

Neonatal hypoxic-ischemic encephalopathy is a neurological disorder that causes damage to cells in the brain in neonates due to inadequate oxygen supply. Brain hypoxia and ischemia due to systemic hypoxemia and reduced cerebral blood flow (CBF) are primary reasons leading to neonatal HIE accompanied by gray and white matter injuries occurring in neonates. Neonatal HIE may cause death in the newborn period or result in what is later recognized as developmental delay, mental retardation, or cerebral palsy (CP). Even though different therapeutic strategies have been developed recently, neonatal HIE remains a serious condition that causes significant mortality and morbidity in near-term, preterm and term newborns and therefore, it remains a challenge for perinatal medicine.

Term neonates suffering from brain injury induced by hypoxia-ischemia (HI) are currently treated with cooling therapy. However, this therapy is only effective in mild cases and associated with adverse outcomes in the preterm newborn, excluding them from any therapy.

Recently, the present inventors have discovered that intravenously administered mesenchymal stem cells (MSC), multipotent adult progenitor cells (MAPC) or extracellular vesicles (EV) derived thereof, were neuroprotective in a translational ovine model of preterm brain injury after global hypoxy-ischemia (HI) [4]. Such therapies require the culturing and administration of eukaryotic cells or products derived therefrom to a subject. This raises issues as to the safety of the treatment as well as concerns regarding costs and logistics of the procedure, in particular when such cells are administered alive.

Hence there is a need for better, safe, reliable and affordable treatments for HIE.

### SUMMARY OF THE INVENTION

It has now been found that hypoxic-ischemic encephalopathy may effectively be treated by administering a composition comprising Annexin A1 or an equivalent thereof. Hence, the invention relates to a composition comprising Annexin A1 or an equivalent thereof, for use in the treatment of neonatal hypoxic encephalopathy due to an ischemic event, wherein the composition comprising Annexin A1 or an equivalent thereof is administered to a neonate within 24 hours after the ischemic event, with the proviso that the composition does not comprise a mesenchymal stem cell (MSC), a multipotent adult progenitor cell (MAPC) or an extracellular vesicle (EV) derived thereof.

### DETAILED DESCRIPTION OF THE INVENTION

We employed an established in vivo ovine model system for HIE as described in example 1. Herein, the umbilical cord was occluded using an inflatable vascular occluder around the umbilical cord to induce global transient hypoxia ischemia (HI).

Our experimental studies as described herein showed that the permeability of the blood-brain barrier (BBB) was increased 4-6 hours after HI accompanied by changes in tight junction protein composition of endothelial cells and

Albumin extravasation.

We found that HI resulted in increased albumin leakage into the brain parenchyma and that this leakage increased continuously after the ischemic event.

This was concluded from the experiments as described herein wherein an analysis of Albumin staining was done on 10 images (200× magnification) of similar sized blood vessels per animal. To evaluate the integrity of the BBB, albumin extravasation was scored with a (+) if positive albumin staining was present in the surrounding cerebral tissue of the blood vessel and a (−) if no albumin was present in the cerebral parenchyma (FIG. 1).

In our experimental model (example 1), we observed an increased leakage of 34% in the HI treated animals compared to the control, seven days after reperfusion (FIG. 1B). These results are provided as a percentage of albumin extravasation indicating leaky blood vessels. This is shown in FIG. 1A, wherein the left panel shows a leaky blood vessel (17% of albumin is outside the vessel) as compared to the right panel from a control sheep wherein 100% of the sheep albumin is contained in the blood vessel. That leakage increased to about 28% at day 3, and 56% at day 7 of reperfusion, respectively. We conclude from these experiments that HI results in a disruption of the blood brain barrier (BBB).

The BBB is composed of endothelial cells which communicate between the peripheral system and cells of the central nervous system such as pericytes, neurons, astrocytes via adherens junctions and transporter structures. This highly specialized structure is crucial for regulating brain homeostasis and protecting the central nervous system (CNS) from potential harmful infiltrating immune cells and inflammatory molecules. Influx of these blood-borne mediators perpetuates neuroinflammation by activation of microglia and subsequent secretion of pro-inflammatory cytokines and reactive oxygen species damaging the developing brain.

We also performed immunohistochemistry on fetal brain sections using antibodies specifically reactive with Annexin A1 (example 2). For the quantification of the intensity of Annexin A1 immunoreactivity (IR) we designed a scoring system (1-3) to evaluate the immunoreactivity intensity of Annexin A1 whereby score 1 comprised minor, score 2 comprised moderate and score 3 comprised intense immunoreactivity. Scoring was complemented by analysis of area fractions, expressed as the percentage of positive staining relative to the total area using a standard threshold intensity, determined with Leica Qwin Pro V 3.5.1. software (Leica, Rijswijk, The Netherlands). Moreover, the thickness of the Annexin A1 positive stained periventricular area was measured with ImageJ software version 1.48. Assessment of Annexin A1 immunoreactivity in microglial cells was determined based on cellular phenotype and staining of adjacent sections with IBA-1 co-localizing with Annexin A1 immunoreactivity.

We found that at 1 day after global HI, Annexin A1 immunoreactivity decreased significantly in blood vessels and ependymal lining cells as compared to controls, whereas after three days and seven days Annexin A1 expression normalized (FIGS. 2A and 2B).

We conclude that the BBB is seriously compromised by the HI and that despite the increased expression of Annexin A1 at day 3, the damage is already done as evidenced by the increasing extravascular presence of endogenous sheep albumin over time. This leaves the practitioner with a window of treatment of at most 3 days, preferably 48 hours, even more preferred 24 or 12 hours, such as 6, 5, 4, 3, 2, or 1 hour or less. Most preferred is a treatment immediately after the ischemic event.

To assess whether the effects on the BBB integrity are mediated by Annexin A1, we used a recognized model for BBB integrity of primary fetal endothelial cells (ECs) isolated from rat brains at postnatal day 3.

A cellular monolayer of endothelial cells (ECs) was cultured on semipermeable filter inserts (Transwell, 3460 Corning). Trans-endothelial electrical resistance (TEER) was measured as an established quantitative readout for barrier integrity as described before (Srinivasan et al., J. Lab Autom. 2015 (2) 107-126) using an Epithelial Voltohmmeter (EVOM2) with two chopstick electrodes, each containing a silver-silver chloride pellet for measuring voltage and a silver pellet for passing current. Measurements of the resistance in ohm (Ω) across the cell layer were made on the semipermeable membrane by placing one electrode in the upper compartment and the other electrode in the lower compartment. Measurements were performed in duplicate per insert and consistently conducted for several days, 30 minutes after culture media was changed and temperature was kept at 37° C. before and between all measurements. Once values plateaued, the membrane reached confluency and further experiments could be performed (baseline measurement).

When ECs reached confluency in the transwells, cells were randomly assigned to oxygen glucose deprivation (OGD) or normoxia conditions. OGD was performed by changing the culture media with DMEM without glucose and glutamine (A1443001 Thermofisher) and exposing ECs to 0% oxygen in a hypoxic chamber at 37 C.° for 4 hours. After 4 hours of normoxia/OGD, medium was changed to culture media and TEER was measured (T0) followed by treatments at the following concentrations and conditions: Annexin A1 (3 μM), FPR1/2 receptor blockers WRW4 (10 μM) and cyclosporine H (1 μM). Retinoic acid (10 μM) was used as a positive control for enhancing BBB integrity (Leoni et al., J. Clin. Invest. 2013 (123(1) 443-454; Lippmann et al., Sci. Rep. 2014 (4) 4160).

Subsequently TEER was measured in all groups at 1 hour, 3 hours, 6 hours, 12 hours and 24 after normoxia/OGD. This setup resulted in following treatment groups (n=2 per experiment): (1) no treatment, (2) Annexin A1, (3) WRW4, (4) WRW4+Annexin A1, (5) cyclosporine H and (6) cyclosporine H+Annexin A1. Normoxia controls were left in normal culture conditions without changing the medium. Cell culture experiments were repeated to test for reproducibility.

Baseline TEER values of our endothelial cells in culture were approximately 150 ohms per insert before experiments continued. At one hour after OGD, TEER values significantly decreased in each treatment group. Subsequently, Annexin A1 treatment steadily increased TEER and values plateaued at 130 ohms (FIG. 3). Strikingly, no treatment or blocking the FPR1 or FPR2 receptor with cyclosporine H and WRW4 resulted in continuous decrease of TEER values down to 100-110 ohms (FIG. 3).

Hence, we have shown herein that Annexin A1 restores the endothelial resistance and/or barrier integrity following oxygen glucose deprivation using an established model for BBB restoration. Altogether, this suggests that strengthening the BBB integrity immediately or soon after the HI attack, prevents brain injury by stimulating endogenous repair mechanisms.

Hence, the invention relates to a composition comprising Annexin A1 or an equivalent thereof, for use in the treatment of neonatal hypoxic encephalopathy due to an ischemic event, wherein the composition comprising Annexin A1 or an equivalent thereof is administered to a neonate within 24 hours after the ischemic event, with the proviso that the composition does not comprise a mesenchymal stem cell (MSC), a multipotent adult progenitor cell (MAPC) or an extracellular vesicle (EV) derived thereof. Annexin A1 may be obtained commercially and is preferably from human origin. Even more preferred is the use of recombinant Annexin A1, such as human recombinant Annexin A1 (Kusters et al., Plos One 10(6) e0130484 DOI:10.1371).

In a preferred embodiment, the composition for use as described above, comprises a pharmaceutically acceptable carrier.

The equivalent of Annexin A1 is preferably selected from the group consisting of human Annexin A1, a truncated Annexin A1 or a chimera with other human Annexins or combinations thereof. The chimera is preferably a fusion protein comprising Annexin A1 and Annexin A5. In a further preferred embodiment, the composition is administered intravenously.

The treatment as described above is preferably performed within 12 hours of the ischemic event, preferably within 6 hours, such as 5, 4, 3, 2, or 1 hour or les, such as immediately after the ischemic event.

Preferably, the Annexin A1 or its equivalent is administered in a dose between 1 μg and 10 mg per kg body weight parenterally as a bolus per 24 hours or as a continuous infusion of a dose between 0.1 μg and 1 mg per kg body weight per hour.

As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

As used herein, the phrase “therapeutic agent” refers to any agent that, when administered to a subject, has a therapeutic effect and/or elicits a desired biological and/or pharmacological effect.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition.

As used herein, the word “preterm” refers to offspring born before the end of the normal period of gestation. For humans, a preterm born baby is a baby born before 37 weeks of gestation. The word “term” refers to offspring born at or after the end of the normal period of gestation. For humans, a term born baby is a baby born at or after 37 weeks of gestation.

### LEGENDS TO THE FIGURES

FIG. 1A: Representative histological images of albumin leakage (arrows) out of blood vessels into the brain parenchyma at 7d after HI (+) and albumin inside the vessel in controls (−).

FIG. 1B: An increased leakage of 34% was observed in the HI treated animals compared to the control, seven days after reperfusion. These results are provided as a percentage of albumin extravasation indicating leaky blood vessels.

FIG. 2: Annexin A1 immunoreactivity in cerebrovasculature (A) and ependymal lining (B) over time after HI. X-axis, time in days (d) Y-axis relative score of Annexin A1 immunoreactivity.

FIG. 3: Annexin A1 improves BBB integrity via the FPR1 and FPR2 receptor. At 0 hour, baseline TEER measurements were taken before initiation of OGD. 4 hours after OGD, cells were treated with Annexin A1 and/or FPR inhibitors and followed up for 3, 6, 12 and 24 hours after treatment. In more detail: fetal rat endothelial cells were objected to OGD for 4 h and at the beginning of reperfusion treated with a composition comprising recombinant Annexin A1. WRW4 and Cyclosporine H, which are FPR2 and FPR1 antagonists. TEER in ohms was measured 3 h, 6 h, 12 h and 24 h after treatment. Time point 0 resembles start of experiment and start of experimental condition (TEER measurement before OGD).

### EXAMPLES

### Example 1: In Vivo Ovine Model

The experimental procedures and study design were in line with institutional guidelines for animal experiments and approved by the Animal Ethics Committee of Maastricht University, The Netherlands. Individual fetuses (n=37) of Texel pregnant ewes randomly received either no occluder (n=18) or an occluder (n=19).

All fetuses were instrumented at 102 days of gestational age (term ˜147 days of gestational age), as previously described (Ophelders et al., Stem Cells Transl. Med. 2016 5(6) 754-763). Concisely, an inflatable vascular occluder was inserted around the umbilical cord for induction of transient global hypoxia ischemia. Further, an umbilical vessel catheter was placed in the femoral artery and brachial vein for measuring blood pressure and administration of MSC-EVs respectively. After a recovery period of 4 days, fetuses were subjected to 25 minutes of sham occlusion or umbilical cord occlusion (UCO) through rapid inflation of the vascular occluder. Fetuses were sacrificed 1 day (n=10), 3 days (n=8) or 7 days (n=19) after (sham) UCO. The investigators performing the (sham) umbilical cord occlusions, tissue sampling and post-mortem analysis were blinded to treatment allocation.

### Example 2: Sample Preparation, Immunohistochemistry and Analysis

After fixation, a predefined region containing the lateral ventricles, periventricular white matter and basal ganglia was embedded in paraffin and serial coronal sections (4 μm) were cut with a Leica RM2235 microtome. Coronal sections were stained for albumin as a marker for BBB leakage, ionized calcium binding adaptor molecule 1 (IBA-1) as a general microglia marker and Annexin A1. First, sections were deparaffinized and rehydrated. Endogenous peroxidase activity was quenched via incubation with 0.3% hydrogen peroxide dissolved in Tris-Buffered Saline (TBS). Antigen retrieval involved boiling tissues in a sodium citrate buffer (pH 6.0) using a microwave oven. Next, sections were incubated overnight with the primary polyclonal rabbit anti-Annexin A1 (AB137745, Abcam; 1:100), anti-albumin (NY11590, Westbury; 1:2000), anti-IBA-1 (019-19741, Wako chemicals; 1:1000) antibody at 4 C.°, followed by incubation with a secondary polyclonal swine anti-rabbit biotin (E0353, Dako; 1:200). The antibody specific staining was enhanced with a Vectastain ABC peroxidase elite kit (PK-6200, Vector Laboratories, Burlingame, Calif.) followed by a 3,3′-diaminobenzidine (DAB) staining. Nuclei were stained with Mayer's hematoxylin.

Analysis of immunohistochemical stainings was done after taking digital images using a Leica DM2000 microscope with Leica Qwin Pro version 3.4.0. software (Leica Microsystems, Mannheim, Germany). Images of Annexin A1 and IBA-1 were taken at a magnification of 100×. Region of interest comprised the blood vessels, ependymal lining cells and white matter including microglial cells stained with IBA-1. Leica QWin Pro V3.4 software was used for processing of the images.

For the quantification of the intensity of Annexin A1 immunoreactivity we designed a scoring system (1-3) to evaluate the immunoreactivity intensity of Annexin A1 whereby score 1 comprised minor, score 2 comprised moderate and score 3 comprised intense immunoreactivity. Scoring was complemented by analysis of area fractions, expressed as the percentage of positive staining relative to the total area using a standard threshold intensity, determined with Leica Qwin Pro V 3.5.1. software (Leica, Rijswijk, The Netherlands. Moreover, the thickness of the Annexin A1 positive stained periventricular area was measured with ImageJ software version 1.48. Assessment of Annexin A1 immunoreactivity in microglial cells was determined based on cellular phenotype and staining of adjacent sections with IBA-1 co-localizing with Annexin A1 immunoreactivity.

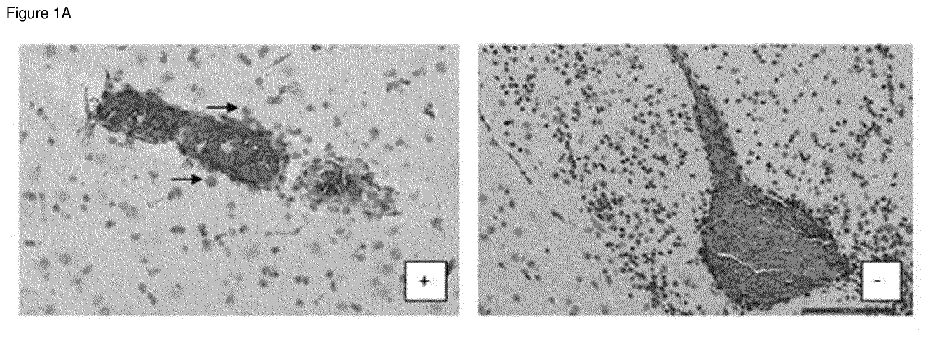

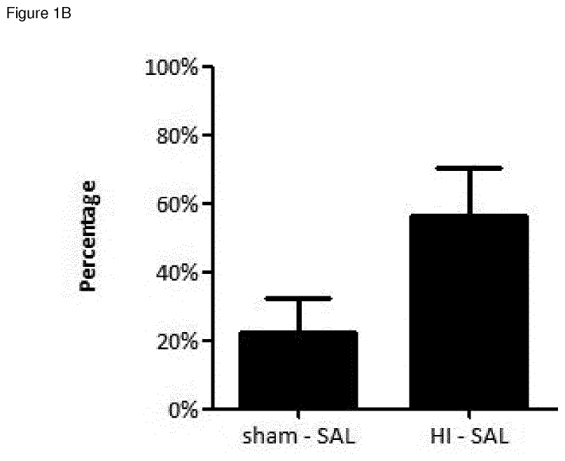

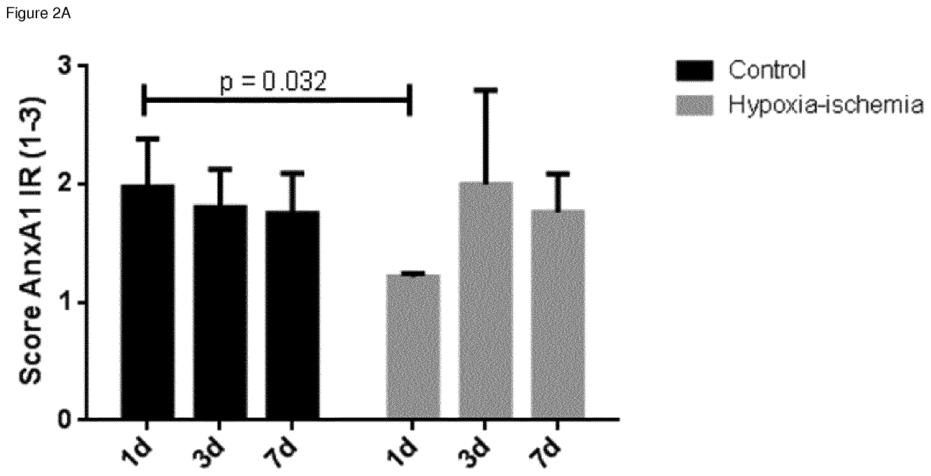

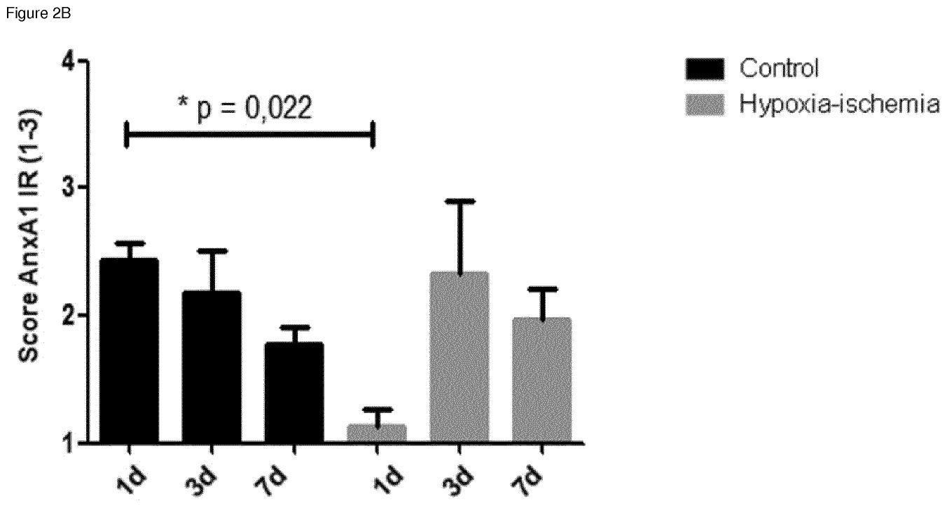

Analysis of Albumin staining was done on 10 images (200× magnification) of similar sized blood vessels per animal. To evaluate the integrity of the BBB, albumin extravasation was scored with a (+) if positive albumin staining was present in the surrounding cerebral tissue of the blood vessel and a (−) if no albumin was present in the cerebral parenchyma (FIG. 1). These results are displayed as a percentage of albumin extravasation indicating leaky blood vessels.

### Example 3:Preparation of Cells for Trans-Endothelial Electrical Resistance (TEER) Analysis

Cells were isolated and cultured as follows. Surplus rat pups sacrificed at postnatal day 3 (P3) by cervical dislocation were received from the Department of Neuroscience of the Maastricht University. The brain developmental stage of rodents on postnatal day 3 is comparable to preterm human infants (Kinney and Volpe; Neurol. Res. Int. 2012, 10.1155/2012/295389. Epub 2012 May 23). Cell isolation protocol was adapted from Bernas et al. (Nat Protoc. 2010 July; 5(7): 1265-1272. Published online 2010 Jun. 10. doi: 10.1038/nprot.2010.76). In short, brains were dissected from the skull and meninges and large vessels were removed before trituration of the tissue by passing the fragments through decreasing pipet tips. Large fragments were filtered out by passing cell suspension through a 500 μM strainer. Cells in the flow-through were collected on a 30 μM strainer and subsequently centrifuged at 51×g for 10 minutes. The resulting pellet was resuspended in DMEM-F12-glutamax (Ser. No. 10/565,018, Thermofisher) supplemented with 10% heat inactivated fetal bovine serum (FBS) (F7524, Sigma), 1% antibiotic-antimycotic solution (A5955, Sigma), 50 μg/mL endothelial cell growth supplement (EGGS) (354006, BD Biosciences), 1 mg/mL heparin (L 6510, Biochrom) and hydrocortisone 500 nM (07904, Stemcell Technologies) and transferred into a T25 flask pre-coated with type-I-collagen (354236, Corning). Culture expansion was allowed for approximately one month to achieve highly confluent endothelial cells showing minimal contamination by pericytes (<5%) as determined by immunocytochemistry.

Characterization of the cells in culture and to assess the purity of the cell population was performed by immunocytochemistry. Cells were grown on glass slides and stained for von Willebrand Factor (vWF) (A0082, Dako), zona-occludens 1 (ZO-1) (61-7300, Invitrogen), Occludin (71-1500, Invitrogen) as endothelial cell markers and α-smooth muscle actin (α-sma) as marker for pericytes (A5228, Sigma). Cells were fixated by incubation in 4% paraformaldehyde (antibodies) or MeOH (antibodies) followed by blocking with bovine serum albumin (BSA), normal goat serum (NGS) or FBS in phosphate buffered saline (PBS). Next, cells were incubated overnight with the primary antibody (1:100/200) at 4 C.°, followed by incubation with the appropriate alexa-fluor labeled secondary antibody (1:200). Nuclei were stained with DAPI and coverslips were mounted using fluorescent mounting medium (Dako).

### Example 4: Trans-Endothelial Electrical Resistance (TEER) Analysis

A cellular monolayer of endothelial cells (ECs) was cultured on semipermeable filter inserts (Transwell) (3460 Corning). TEER was measured as an established quantitative readout for barrier integrity (Srinivasan et al., J. Lab Autom. J Lab Autom. 2015 April; 20(2): 107-126, Published online 2015 Jan. 13. doi: 10.1177/2211068214561025) using an Epithelial Volt-ohmmeter (EVOM2) with two chopstick electrodes, each containing a silver-silver chloride pellet for measuring voltage and a silver pellet for passing current. Measurements of the resistance in ohm (Q) across the cell layer were made on the semipermeable membrane by placing one electrode in the upper compartment and the other electrode in the lower compartment. Measurements were performed in duplo per insert and consistently conducted for several days, 30 minutes after culture media was changed and temperature was kept at 37° C. before and between all measurements. Once values plateaued, the membrane reached confluency and further experiments could be performed (baseline measurement).

When ECs reached confluency in the transwells, cells were randomly assigned to oxygen glucose deprivation (OGD) or normoxia conditions. OGD was performed by changing the culture media with DMEM without glucose and glutamine (A1443001 Thermofisher) and exposing ECs to 0% oxygen in a hypoxic chamber at 37 C.° for 4 hours. After 4 hours of normoxia/OGD, medium was changed to culture media and TEER was measured (TO) followed by treatments at the following concentrations and conditions: Annexin A1 (3 μM), FPR1/2 receptor blockers WRW4 (10 μM) and cyclosporine H (1 μM). Retinoic acid (10 μM) was used as a positive control for enhancing BBB integrity (Leoni et al., J. Clin. Invest. 2013 (123(1) 443-454; Lippmann et al., Sci. Rep. 2014 (4) 4160).

Subsequently TEER was measured in all groups at 1 hour, 3 hours, 6 hours, 12 hours and 24 after normoxia/OGD. This setup resulted in following treatment groups (n=2 per experiment): (1) no treatment, (2) Annexin A1, (3) WRW4, (4) WRW4+Annexin A1, (5) cyclosporine H and (6) cyclosporine H+Annexin A1. Normoxia controls were left in normal culture conditions without changing the medium. Cell culture experiments were repeated to test for reproducibility.

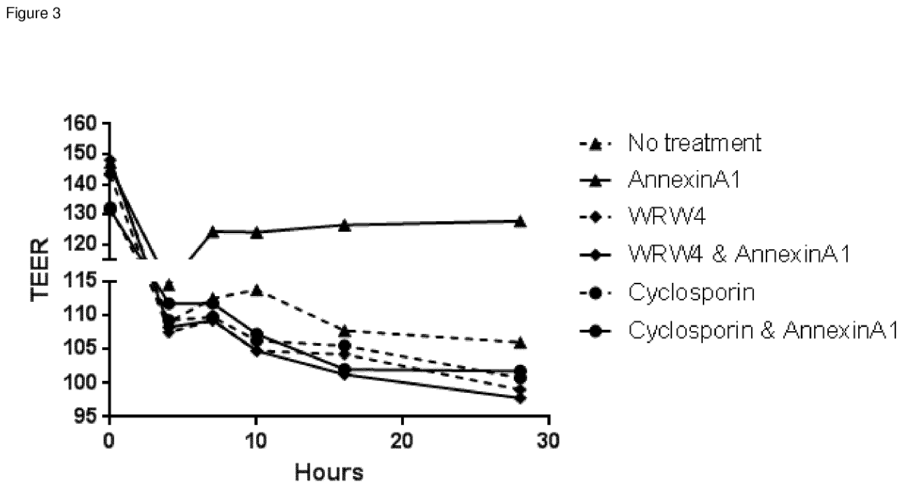

Baseline TEER values of our endothelial cells in culture were approximately 150 ohms per insert before experiments continued. At one hour after OGD, TEER values significantly decreased in each treatment group. Subsequently, Annexin A1 treatment steadily increased TEER and values plateaued from 12 h onwards at 130 ohms. Strikingly, no treatment or blocking the FPR1 or FPR2 receptor with cyclosporine H and WRW4 resulted in continuous decrease of TEER values down to 100-110 ohms (FIG. 3).

### Example 5: Statistical Analysis

Immunohistochemistry: All values are shown as mean with 95% confidence interval (CI) or standard deviations (SD). Comparison between different experimental groups was performed with analysis of variance (ANOVA).

Data from TEER measurements were obtained from 2 independent experiments each run in n=2 per treatment group. Resistance across the endothelial cell layer on the semipermeable membrane (Q) times effective area of the semipermeable membrane (cm2). These adjusted resistance measurements were expressed as a ratio to the corresponding mean adjusted resistance measurement. As a result, normalized values were compared between the different experimental groups. Data was presented using GraphPad Prism 5 and tested with an unpaired sample t-test for significance.

Statistical analysis was performed with IBM SPSS Statistics Version 22.0 (IBM Corp., Armonk, N.Y., USA; SPSS) graphical design was performed using GraphPad Prism 5. Exact p-values are reported and statistical significance was accepted at p<0.05.

### REFERENCES

1. Ferriero D M. Neonatal brain injury. N Engl J Med. Nov. 4 2004; 351(19):1985-95.
2. Perlman J M. Brain injury in the term infant. Semin Perinatol. December 2004; 28(6):415-24.
3. Grow J, Barks J D. Pathogenesis of hypoxic-ischemic cerebral injury in the term infant: current concepts. Clin Perinatol. December 2002; 29(4):585-602, v.
4. Jellema et al., PLoS ONE 8(8) (2013) e73031.
5. Lai et al., Stem Cell Res. (2010) 4: 214-222.
6. Ludwig et al., Int. J. Biochem Cell Biol (2012) 44:11-15.
7. Jellema et al., J. Neuroinflamm. (2013) 10: 13.
8. Kumar et al., Pediatrics (2008) 122(3): e722-727

1. A composition comprising Annexin A1 or an equivalent thereof, for use in the treatment of neonatal hypoxic encephalopathy due to an ischemic event, wherein the composition comprising Annexin A1 or an equivalent thereof is administered to a neonate within 24 hours after the ischemic event, with the proviso that the composition does not comprise a mesenchymal stem cell (MSC), a multipotent adult progenitor cell (MAPC) or an extracellular vesicle (EV) derived thereof, wherein the equivalent is selected from the group consisting of human Annexin A1, a truncated Annexin A1 and a chimera of human Annexin A1 with at least one other human Annexin.

2. The composition of claim 1 wherein the composition comprises a pharmaceutically acceptable carrier.

3. The composition of claim 1 wherein the equivalent is a chimera comprising Annexin A1 and Annexin A5.

4. The composition of claim 1 wherein the treatment comprises intravenous administration.

5. The composition of claim 1 wherein the treatment occurs within 12 hours of the ischemic event.

6. The composition of claim 1 wherein the Annexin A1 or an equivalent thereof is administered in a dose between 1 μg and 10 mg per kg body weight.

7. The composition of claim 6 wherein the Annexin A1 or an equivalent thereof is administered parenterally as a bolus per 24 hours.

8. The composition of claim 6 wherein the Annexin A1 or an equivalent thereof is administered as a continuous infusion of a dose between 0.1 μg and 1 mg per kg body weight per hour.

9. The composition of claim 1 wherein the composition does not contain an intact cell.

10. The composition of claim 9 wherein the cell is a stem cell.

11. The composition of claim 10 wherein the stem cell is a mesenchymal stem cell.
